(19) United States
(12) Patent Application Publication
(10) Pub. No.: US 2005/0042675 A1
(43) Pub. Date: Feb. 24, 2005

(54) Solution for preparing stool specimens for diagnostic purposes

(75) Inventors: Meret Lakner, Munchen (DE); Andreas True, Pretoria (ZA); Sonja Dehnert, Munchen (DE); Christian Reiter, Karlsfeld (DE); Gerhard Cullmann, Krottelbach (DE); Petra Heppner, Pullach (DE); Eva Haindl, Pentenried (DE); Andreas Finck, Munchen (DE); Helmut Benesch, Woreshofen (DE); Achim Ringeis, Grafelfing (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Connex Gesellschaft zur Optimierung von Forschung und Entwicklung MBH, Martinsried (DE)

(21) Appl. No.: 10/362,976

(22) PCT Filed: Aug. 31, 2001
(86) PCT No.: PCT/EP01/10067

(30) Foreign Application Priority Data
Sep. 1, 2000 (DE) 100 43 161.5

(51) Int. Cl. G01N 33/53

(52) U.S. Cl. 435/7.1

(57) ABSTRACT

The present invention relates to a new solution for the preparation of stool samples for diagnostic tests. The solution allows samples to be prepared simply before use in an immunological detection process and also provides a high level of sensitivity, specificity and reproducibility of the tests. The solution must contain at least one buffer substance, one detergent and one blocking reagent. The Invention also relates to a process for the analysis of a stool sample for diagnostic purposes using the solution according to the invention.

The present invention relates to absolution for the preparation of stool samples for diagnostic tests and a process for the analysis of a stool sample for diagnostic purposes.

Within diagnostics, stool samples are routinely tested for the presence of bacteria, viruses, parasites and other organisms. With immunological tests, for example, antigens of the corresponding organisms can be detected. Diagnostic detection processes of this type comprise the taking of stool samples, usually a few steps for the preparation of the stool sample and the actual immunological test process in which the presence or absence of the agent to be detected, e.g. an antigen is demonstrated on the basis of a reaction, such as a colour reaction.

In contrast to invasive techniques, such as endoscopy and biopsy, which are generally stressful for the organism and are often also associated with a high equipment requirement and a health risk non-invasive techniques such as stool sampling and subsequent analysis provide a simple opportunity to detect organisms that live in the digestive tract.

The use of immunological test procedures to analyse stool samples, however, can be difficult for several reasons: Handling the stool samples is unpleasant and the preparation of stool samples is laborious, complex and time-consuming. In order to be able to use stool samples in an immunological test process, impurities which could disrupt the test procedure must first be removed from them. Normally the preparation of stool samples therefore involves several stages such as dilution, centrifugation or flitration, for example.

U.S. Pat. No. 5,198,365, for example, describes a method for stool preparation through sample dilution by a factor of 10 to 100. This type of sample dilution, however, has a disadvantageous effect on the sensitivity of the test and the incubation times when carrying out the test. Vellacott et al. (Lancet 32 (1): 249 (1981)) describe an immunological test for the detection of occult blood in the stool in which a centrifugation phase is required for the sample preparation. Hasan et al. (J. Clin. Micro. 32: 249 (1994)) describe an immuno-diagnostic test for the detection of Vibric cholera in clinical samples. In this process, the sample has to be purified first using a separate filter.

Accordingly, the state of the art describes procedures for the diagnosis, e.g., of Entamoeba histolytica (Haque (1993), J. Infect. Dis. 167: 247-9), enterohaemorrhagic Escherichia coli (EHEC; Park (1995), J. Clin. Microbiol. 34: 988-990), torovirus-like particles (Koopmans (1993), J. Clin. Mlcrobiol. 31: 2738-2744) or Taenia saginata (Machnicka (1996), Appl. Parasitol. 37: 106-110) from stool.

The element that these processes have in common is that one or several separate process stages for sample treatment precede the actual test. Samples are generally prepared by suspending the stool sample in a suitable suspension buffer. The composition of this buffer has a major influence on the sensitivity and specficity of the test. Excluding the detection of falsely positive samples often proves to be particularly problematic. The detection of falsely positive samples depends essentially on the composition of the sample buffer components. In addition, the protein content of the sample affects the viscosity of the sample suspension and thus influences the flow behaviour of the sample suspension.

Sample preparation should be optimised in terms of the following: high reproducibility, high sensitivity, high specificity and low viscosity.

The present invention was based on the technical problem of providing a solution that allows the simplest possible sample preparation whilst giving the maximum reproducibility, sensitivity and specificity of the proof.

A further technical problem was in providing a process as simple as possible for analysing stool samples for diagnostic purposes. The process should guarantee the simplest possible sample handling whilst retaining maximum sensitivity, specificity and reproducibility of the results obtained.

The first problem is solved according to the invention by a solution for the preparation of a stool sample for diagnostic purposes containing at least one buffer substance, at least one detergent and at least one blocking reagent.

The second problem is solved according to the invention by a process for the analysis of stool samples for diagnostic purposes covering the following steps:
(a) bringing the sample into contact with a solution according to the invention;
(b) carrying out an immuno assay with the sample treated under step (a); and
(c) taking a measurement signal which was obtained within the scope of the immuno essay.

The solution according to the invention is particularly suitable for the preparation of stool samples in which the diagnosis of a Helicobacter pylori infection is to be carried out. Basically, however, preparing the sample using the solution according to the invention allows any pathogens in the digestive tract to be detected.

In a particularly preferred embodiment, the buffer substance is selected from PBS, TBS, glycine buffer (0.1 M glycine, 140 mM NaCl), HEPES ([4-(2-hydroxyethly)-piperazino]-ethane sulfonic acid), MOPS (3morphollno-1-propane sulfonic acid), whereby PBS is particularly preferred.

In a further preferred embodiment, the pH of the solution is in a range from 7.0 to 8.0, preferably 7.2 to 7.7, whereby optimum results for the detection of H. pylori are obtained with a pH from 7.3 to 7.5.

In a further preferred embodiment, the detergent is a zwitterionic detergent that is preferably selected from Chaps (3-[(3-chloramidopropyl)-dimethylammonium]-1-propane sulfonate) and Zwittergent® (N-dodecyl-N,N-dimethyl-3-ammonio-1-propane sulfonate), whereby Chaps is very particularly preferred.

Detergents are essential for breaking down the sample material. In the event of an immunological detection, they release antigen structures (epitopes) in order to allow, in this way, a binding of the antibodies used to the antigen to be detected. Normally, non-ionic detergents such as Triton®, Tween® etc. are used. However, within the framework of the present invention, it has been found, surprisingly, that the use of a zwitterionic detergent has major advantages over the use of non-ionic detergents in that, for example, the detection of falsely positive samples is considerably reduced when the zwitterionic detergent is used. The preferred detergent, Chaps, has further advantages over further detergents such as Triton® or Tween®: compared with these detergents, Chaps has fewer denaturing properties (Hjelmeland; U.S. Pat. No. 4,372,888). The surface proteins thus, during the breakdown (during sample preparation), remain basically as intact complexes in the membrane. The surface proteins are fragmented and denatured to a lesser extent. The probability of obtaining antigen epitopes during the breakdown which are sufficient for immunological detection is therefore increased. In particular, with immunological detection in sample material that has passed through the digestive tract, the enzymatic degradation of the antigen can already cause a high degree of fragmentation of the sample material. The most efficient retention of the residual epitopes left is therefore extremely desirable.

In a further preferred embodiment, the detergent is in a concentration of 0.01 to 1.5% (v/v), preferably in a concentration of 0.05 to 0.3% (v/v) and, very particularly preferred, in a concentration of 0.1 to 0.15% (v/v).

The blocking reagent contained in the solution according to the invention may be an animal serum or a protein mixture. In a preferred embodiment, the solution according to the invention will contain a blocking reagent that is selected from mouse serum, bovine serum, human serum, rabbit serum, pig serum, foetal calf serum, BSA, skimmed milk powder and peptone, whereby mouse serum is particularly preferred. Mouse serum should be particularly preferred if an immuno assay is carried out in which mouse antibodies are used to detect an antigen. The use of serum from the same species as the antibodies used for detection (e.g. mouse serum in combination with mouse antibodies) has been found to be particularly advantageous for the blocking of non-specific compounds, since the potential non-specific compounds can be almost completely saturated/blocked through serums of the same species. The higher saturation of the non-specific compounds in turn increases the sensitivity/specificity of the test.

In a further preferred embodiment, the blocking reagent is in a concentration of 0.05 to 5%, preferably in a range from 0.1 to 1%, and very particularly preferred in a range from 0.4 to 0.6% in the solution according to the invention. The percentage concentration information is given as “% (v/v)” for liquid blocking reagents such as mouse serum, bovine serum, human serum, rabbit serum, pig serum and foetal calf serum and as “% (w/v)” for solid blocking reagents such as BSA, skimmed milk powder and peptone.

Conventional solutions for sample preparation often show a very high level of serum. Often, concentrations of up to 50% serum are used. However, the effect of the high serum content is a high viscosity of the prepared sample, thus making handling more difficult. This problem does not occur if the solution according to the invention is used: If the solution according to the invention contains serum as the blocking reagent, the serum content in the solution is lower than with conventional solutions for sample preparation. This reduces the viscosity of the prepared sample, which is a particular advantage for handling during sample preparation. The flow behaviour is improved considerably, which means that the sample buffer is particularly suitable for use in ELISA or immune chromatography tests (test strips). Particularly with applications using a test strip, a low viscosity leads to better running behaviour, which allows a higher throughput of samples per unit of time. Through using serum of the same species as the detection antibodies, such as mouse serum in combination with mouse antibodies, the level of serum in the conjugate buffer can also be reduced.

In a further preferred embodiment, the solution contains further additives such as sample stabilising agents. The aim of such agents is to ensure that the sample is still suitable even after a long period of storage for immunological test reactions to be carried out and does not provide any distorted results. These stabilisers include phenol and antibiotics, such as gentamicin sulfate and Proclin® 300.

The solution according to the invention is also particularly advantageous in that it can be used as a basic buffer both for the conjugate (marked detection antibodies) and for the positive and negative control, which means that the test process can be made much simpler.

In a further preferred embodiment, the solution also contains a complexing agent for metal ions, preferably EDTA or EGTA EDTA is particularly preferable. Preferably, the complexing agent will be in a concentration of 1 mM in the solution.

The solution according to the invention is preferably used within the framework of a test kit, whereby the test kit contains all the reagents to carry out the immuno assay. These include, for example, test strips or plates with recesses (e.g. micro titre plates), in which the reactions proceed, plus the various detection reagents including the antibodies used and if necessary substrates for detection using ELISA. Preferably, all the reagents will be contained in a single pack unit with separate sub-compartments.

In the process according to the invention for the analysis of a stool sample for diagnostic purposes, the sample is brought into contact with the solution according to the invention. Here, the sample is generally suspended in the solution according to the invention. After distributing the sample in the solution as homogeneously as possible, the latter is then subjected to an immuno assay and after the completion of the immuno assay, the measurement signal produced is recorded. Before the immuno assay, the particular sample elements can be removed by centrifuging or filtration.

In a preferred embodiment, the immuno assay is carried out as an ELISA. Here, the suspended sample is placed in a container which contains the antigen-specific reagents. Preferably, within the scope of this ELISA, a sandwich complex is formed from the antigen to be detected and antigen-specific antibodies. In an alternative embodiment, the suspended sample is placed on a filter strip which already contains the corresponding detection reagents at previously determined zones. Carrying out the immuno assay on a filter has the advantage that undesired particular sample elements of the antigen in question are separated before testing for it. A filter may be pre-attached in addition, to give better separation.

The following examples explain the invention.

### EXAMPLES

A Comparison of Various Solutions for Sample Preparation in a One-step ELISA

For the test, stool samples from patients from ten different clinics or gastro-enterological practices were available, which had been diagnosed as H. pylori negative (G0) or H. pylori positive (G4) using ¹³C-urea breath test and/or histological examinations of stomach biopsies.

H. pylori Stool Sandwich EUSA (One-step Test)

The ELISA plate (MaxiSorb Lock well; Nunc) was coated over night at 2-8° C. with 100 μl of a mAK solution (2.0 μg HP25.2 m/2H10 (Connex, Martinsried) per ml carbonate buffer, 0.1 M, pH 9.5). The ELISA plates coated in this way were washed 2× with PBS. To block the free bonding points, 200 μl blocking buffer (0.3% (w/v) BSA; 20% (w/v) sorbitol in PBS) were added per recess and incubated over night at 2-8° C. The saturated plates were drawn off, dried over night at 28° C. in a circulating air drying cabinet and then stored at 2-8° C.

Patient stool was suspended 1:5 (0.1 g stool sample+500 μl sample buffer) in the solution according to the invention (75 mM PBS+0.5% mouse serum+1 mM EDTA+0.05% Proclin® 300+50 μg/ml gentamicin sulfate+10 mM phenol+0.1% (v/v) Chaps) for approx. 30 sec (Vortex) and then centrifuged for 5 minutes at 5000 g. Per recess, 50 μl of the residue (double to triple determination) were applied to the plate.

Then 50 μl of the POD-marked antibody HP25.2 m/2H10-POD (Connex, Martinsried) diluted in the sample buffer were added directly to the stool suspension. The plates were incubated for 1 hour at room temperature on the shaker (level 4-5).

After washing four times with washing buffer (75 mM PBS, 0.25% (v/v) Tween®, the perodixase substrate TMB (single-component substrate of neogen) was added (100 μl/recess). After 10 minutes, the enzyme reaction was stopped by the addition of 1 N hydrochloric acid (100 μl/recess). The colour intensity was then measured at 450 nm against the reference wavelength of 630 nm.

### Example 1

Comparison of the Signal/background Relationship of Various Solutions for Sample Preparation

TABLE 1
shows a comparison of various solutions using selected
samples which proved particularly difficult
for detection purposes.

| | HP25.2 m/2H10 (2.0 μg/ml) | | | |
|---|---|---|---|---|
| Capture | HP25.2 m/2H10-POD (200 ng/ml) | | | |
| antibodies | | r-biopharm | solution | PBS + skimmed |
| Detection | | sample | acc. to | milk powder (2% |
| antibodies | | buffer | invention | (w/v)) |
| Sample buffer | Sample | OD | OD | OD |
| CX1002 | G0 | 2.026 | 0.534 | 0.678 |
| CX2028 | G0 | 0.059 | 0.034 | 0.090 |
| CXT0069-1 | G0 | 0.079 | 0.021 | 0.032 |
| CXT0071-1 | G0 | 0.348 | 0.030 | 0.051 |
| CXT0072-1 | G0 | 0.079 | 0.036 | 0.052 |
| CXT0073-1 | G0 | 0.044 | 0.036 | 0.076 |
| CXT0074-1 | G0 | 0.125 | 0.028 | 0.058 |
| CXT1038 | G4 | 0.100 | 0.040 | 0.074 |
| CXT0053-1 | G4 | 4.458 | 4.247 | 4.285 |
| CXT0058-1 | G4 | 0.221 | 0.146 | 0.192 |
| CXT0077-1 | G4 | 0.264 | 0.180 | 0.198 |
| Total G4 | | 5.607 | 4.714 | 4.831 |
| Total G0 | | 2.760 | 0.719 | 1.037 |
| Ag-DL in ng/ml | | 0.33 | 0.33 | 0.11 |
| Background | | 0.102 | 0.08 | 0.035 |

r-biopharm solution available from r-biopharm, Darmstadt
OD: optical density
DL: detection limit

Result: 75 mM PBS+0.5% mouse serum+1 mM EDTA+0.05% (w/v) Proclin® 300+30 μg/ml gentamicin sulfate+0.1% (v/v) Chaps showed the best signal/background ratio of the 3 buffers studied.

### Example 2

Influence of Various Chaps Concentrations in the Solution According to the Invention on Signal/background Ratio and the Sensitivity of the Test

Result: The table shows that the addition of Chaps (0.1-0.25%
(v/v)) produces a positive G0/G4 signal ratio.

| Capture | HP25.2 m/2H10 1.5 μg/ml | | | |
|---|---|---|---|---|
| antibodies | HP25.2 m/2H10-POD 0.2 μg/ml) | | | |
| Detection | | 0.1% (v/v) | 0.25% (v/v) | 0.5% (v/v) |
| antibodies | 0% Chaps | Chaps | Chaps | Chaps |
| Antigen detection | 0.3 | 0.3-1 | 0.3 | 0.1-0.3 |
| [ng/ml] | | | | |
| Background | 0.15 | 0.067 | 0.04 | 0.026 |
| HO delta OD | | | | |
| delta OD at 9 | 0.759 | 0.89 | 0.787 | 1.267 |
| ng/ml <60 kDA | | | | |
| Patient samples | | | | |
| CXT 73 G0 | 0.12 | 0.03 | 0.04 | 0.06 |
| CXT 54 G0 | 0.11 | 0.03 | 0.04 | 0.05 |
| CX 60 G0 | 0.12 | 0.065 | 0.04 | 0.05 |
| CXT 63 G0 | 0.07 | 0.05 | 0.045 | 0.05 |
| CXT 62 G0 | 0.03 | 0.07 | 0.03 | 0.03 |
| CXT 64 G4 | 0.51 | 0.94 | 0.9 | 0.5 |
| CXT 58-3 04 | 0.2 | 0.38 | 0.27 | 0.21 |

Result; The table shows that the addition of Chaps (0.1-0.25% (v/v)) produces a positive G0/G4 signal ratio.

1. Process for the analysis of a stool sample for the diagnosis of an H. pylori infection comprising the following steps:
a) bringing the sample into contact with a solution that has a pH in a range from 7.0 to 8.0 containing:
at least one buffer substance selected from PBS, glycine buffer (0.1 M glycine, 140 mM NaCI), HEPES ([4-(2-hydroxyethyl)-piperazino]-ethane sulfonic acid) and MOPS (3-Morpholino-1-propane sulfonic acid);
the zwitterionic detergent Chaps (3-[(3-chloramidopropyl)-dimethylammonium]-1-propane sulfonate) in a concentration of 0.01 to 1.5% (v/v), preferably in a range from 0.05 to 0-3% (v/v), very particularly preferred in a range from 0.1 to 0.15% (v/v); and
the blocking reagent mouse serum in a concentration of 0.05 to 5%, preferably in a range from 0.1 to 1%, very particularly preferred in a range from 0.4 to 0.6%;
if necessary, gentamicin sulfate and/or Proclin TM 300 as the stabiliser;
and if necessary a complexing agent, preferably selected from EDTA and EGTA, particularly preferred EDTA, preferably in a concentration of 1 mM;
b) carrying out an immuno assay with the sample treated according to step a); and
c) taking of a measurement signal that was obtained within the scope of the immuno assay.

2. Process according to claim 1, characterized in that the immuno assay is an ELISA.

3. Process according to claim 1, characterized in that the stool sample treated with the solution is applied to a filter strip.
